(19) United States
(12) Patent Application Publication
(10) Pub. No.: US 2023/0033205 A1
(43) Pub. Date: Feb. 2, 2023

(54) AUTOMATED SYSTEM FOR CONTROLLING THE BLOOD GLUCOSE LEVEL

(71) Applicant: Commissariat à L'Énergie Atomique et aux Énergies Alternatives, Paris (FR)

(72) Inventors: Emma Villeneuve, Grenoble Cedex 9 (FR); Eléonore-Maeva Doron, Grenoble Cedex 9 (FR)

(73) Assignee: Commissariat à L'Énergie Atomique et aux Énergies Alternatives, Paris (FR)

(21) Appl. No.: 17/779,509

(22) PCT Filed: Nov. 18, 2020
(86) PCT No.: PCT/EP2020/082600

§ 371 Date: May 24, 2022

(30) Foreign Application Priority Data
Nov. 27, 2019 (FR) FR1913336

(51) Int. Cl. G16H 20/17

(52) U.S. Cl. CPC G16H 20/17; A61M 2230/201

(57) ABSTRACT

An automated blood glucose regulation system, including: a blood glucose sensor; an insulin injection device; and a processing and control unit, wherein the processing and control unit is configured to implement a method of management of undeclared meals.

The present patent application claims the priority benefit of French patent application FR19/13336 which is herein incorporated by reference.

### TECHNICAL BACKGROUND

The present application relates to the field of automated blood glucose regulation systems, also called artificial pancreases.

### PRIOR ART

Automated blood glucose regulation systems, also called artificial pancreases, enabling to automatically regulate the insulin inputs of a diabetic user based on their glycemia (or blood glucose) history, on their meal history, on their insulin injection history, have already been provided.

Examples of regulation systems of this type are particularly described in international patent applications No WO2018/055283 (DD16959/B15018), No WO2018/055284 (DD17175/B15267), No WO2019/016452 (DD17609/B15860), and No WO2019/180341 (DD18479/B16770), and in French patent applications No 18/56016 of Jun. 29, 2018 (DD18587/B16893), No 18/00492 of May 22, 2018 (DD18480/B16894), No 18/00493 of May 22, 2018 (DD18588/B16895), No 18/73812 of Dec. 21, 2018 (DD18986/B17521), and No 19/08457 of Jul. 25, 2019 (DD19664/B18647) previously filed by the applicant.

It would be desirable to be able to improve the performances of known artificial pancreases, and particularly to be able to further limit risks of placing the user in a hyperglycemia or hypoglycemia situation.

The management of meals not declared in advance by the user is here more particularly considered.

### SUMMARY OF THE INVENTION

An embodiment provides an automated blood glucose regulation system, comprising:
a blood glucose sensor;
an insulin injection device; and
a processing and control unit,
   wherein the processing and control unit is configured to implement a method of management of undeclared meals, this method comprising the steps of:

a) detecting, at a time t0, an event likely to correspond to a meal non-declared by a user;

b) when an event is detected at step a), determining, based on a first table generated by training from a history of the user's data, a probability for a meal to have been taken by the user within a period T_ANT of predetermined duration preceding time t0;

c) if the probability determined at step b) is greater than a threshold TH, determining, based on the first table, an estimated time slot of meal ingestion by the user within period T_ANT and, based on a second table generated by training from a history of the user's data, an estimated size of the meal, and then activating a meal management module of the regulation system and transmitting to said module the estimated time slot and size of the meal.

According to an embodiment:
the first table is formed of a series of probability values, each corresponding to a percentage of times that a meal has been declared by the user within a determined time interval of a determined time cycle, during a training phase comprising a plurality of occurrences of said time cycle; and
the second table is formed of a series of meal size values, each corresponding to the average size of the meals declared by the user within each time interval of said time cycle during the training phase.

According to an embodiment, said time cycle is divided into a plurality of time intervals, the number of values of the first table and the number of values of the second table being equal to the number of time intervals of the time cycle.

According to an embodiment, the processing and control unit is configured to, if the probability determined at step b) is lower than threshold TH, implement a blood glucose regulation method non-specific to meals.

According to an embodiment, the system further comprises a user interface device coupled to the processing and control unit.

According to an embodiment, the processing and control unit is configured to, when an event is detected at step a), implement, before step b), a first step of interrogation of the user, by means of the user interface device, to ask them whether they have had an undeclared meal within period T_ANT.

According to an embodiment, the processing and control unit is configured to:
if the user answers negatively to the first interrogation, implement a blood glucose regulation method non-specific to meals;
if the user answers positively to the first interrogation, implement a second step of interrogation of the user, by means of the user interface device, to ask them the time and the size of said undeclared meal taken during period T_ANT; and
if the user does not answer to the first interrogation, implement step b) and then step c).

According to an embodiment, the processing unit is configured to:
if the user answers to the second interrogation, activate the meal management module of the regulation system and transmit to said module the time and the size declared by the user as an answer to the first interrogation; and
if the user does not answer to the second interrogation, determine, based on the first table, an estimated time slot of meal ingestion by the user within period T_ANT and, based on the second table, an estimated size of the meal, and then activate the meal management module of the regulation system and transmit to said module the estimated time slot and size of the meal.

According to an embodiment, the processing and control unit is configured to, at step c), determine, by means of the meal management module, an insulin bolus to be injected to the user according to the estimated meal size.

According to an embodiment, the processing and control unit is configured to, at step c), weigh the bolus with an aggressiveness factor which is a function of the probability determined at step b).

According to an embodiment, the first and second tables are stored in a memory circuit of the processing and control unit.

### BRIEF DESCRIPTION OF THE DRAWINGS

The foregoing features and advantages, as well as others, will be described in detail in the following description of specific embodiments given by way of illustration and not limitation with reference to the accompanying drawings, in which:

FIG. 1 schematically shows, in the form of blocks, an example of an automated system for regulating a subject's blood glucose according to an embodiment;

FIG. 2 is a diagram illustrating an example of an automated blood glucose regulation method capable of being implemented by the system of FIG. 1;

FIG. 3 shows an example of a first lookup table that may be used for the implementation of the method of FIG. 2; and

FIG. 4 shows an example of a second lookup table that may be used for the implementation of the method of FIG. 2.

### DESCRIPTION OF THE EMBODIMENTS

Like features have been designated by like references in the various figures. In particular, the structural and/or functional features that are common among the various embodiments may have the same references and may dispose identical structural, dimensional and material properties.

For the sake of clarity, only the steps and elements that are useful for an understanding of the embodiments described herein have been illustrated and described in detail. In particular, the blood glucose measurement devices and the insulin injection devices of the described regulation systems have not been detailed, the described embodiments being compatible with all or most known blood glucose measurement and insulin injection devices. Further, the implementation of the processing and control unit of the described regulation systems has not been detailed, the forming of such a processing and control unit being within the abilities of those skilled in the art based on the functional indications of the present disclosure.

Unless specified otherwise, the expressions “around”, “approximately”, “substantially” and “in the order of” signify within 10%, and preferably within 5%.

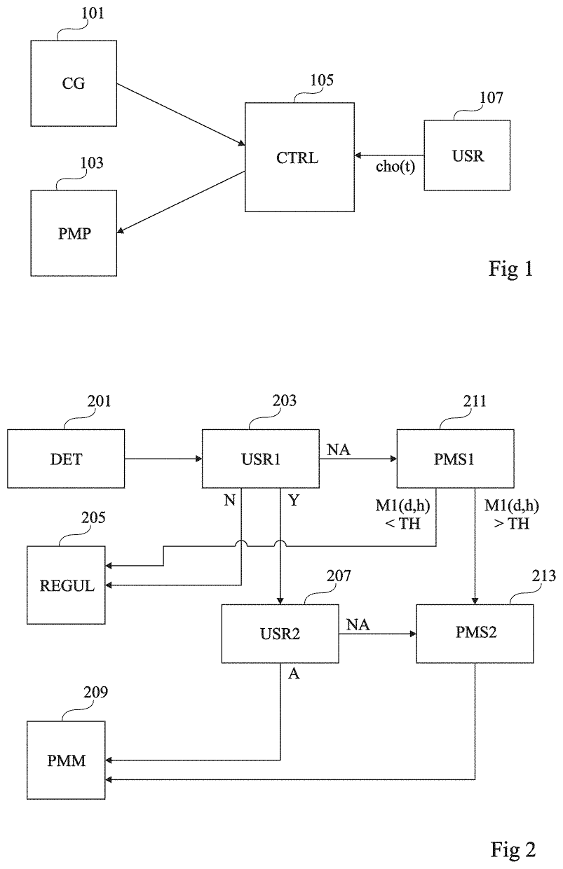

FIG. 1 schematically shows in the form of blocks an example of an embodiment of an automated system for regulating a user's blood glucose.

The system of FIG. 1 comprises a sensor 101 (CG) adapted to measuring a quantity representative of the user's blood glucose, for example, the glucose concentration in the interstitial liquid, which will be called blood glucose hereafter for simplification. In normal operation, sensor 101 may be permanently positioned on or inside of the user's body, for example, at the level of their abdomen or at the level of their arm. Sensor 101 is for example a CGM-type (“Continuous Glucose Monitoring”) sensor, that is, a sensor capable of measuring, continuously or at a relatively high frequency (for example, at least once every twenty minutes and preferably at least once every five minutes) the user's blood glucose. Sensor 101 is for example a subcutaneous blood glucose sensor.

The system of FIG. 1 further comprises an insulin injection device 103 (PMP), for example, a subcutaneous injection device. Device 103 is for example, an automatic injection device of insulin pump type, comprising an insulin reservoir connected to an injection needle implanted under the user's skin, and the pump may be electrically controlled to automatically inject determined insulin doses at determined times. In normal operation, injection device 103 may be permanently positioned inside or on the user's body, for example at the level of their abdomen.

The system of FIG. 1 further comprises a processing and control unit 105 (CTRL) connected on the one hand to blood glucose sensor 101, for example, by a wire link or by a radio (wireless) link, and on the other hand to injection device 103, for example, by wire or radio link. In operation, processing and control unit 105 is capable of receiving the data relative to the user's blood glucose measured by sensor 101, and of electrically controlling device 103 to inject to the user determined insulin doses at determined times. In this example, processing and control unit 105 is further capable of receiving, via a user interface 107 (USR), data cho(t) representative of the time variation of the quantity of glucose ingested by the user.

Processing and control unit 105 is capable of determining the insulin doses to be injected to the user by taking into account, in particular, the history of the blood glucose measured by sensor 101, the history of the insulin injected by device 103, and the history of carbohydrate ingestion by the user. To achieve this, processing and control unit 105 comprises a digital calculation circuit (not detailed), for example comprising a microprocessor. Processing and control unit 105 is for example a mobile device carried by the user all along the day and/or the night, for example, a smart phone-type device configured to implement a regulation method of the type described hereafter.

Processing and control unit 105 is for example configured to, outside of meal periods, implement an automated MPC-type (“Model-based Predictive Control”) regulation method, also called predictive control method, where the regulation of the administered insulin dose takes into account a prediction of the future trend of the user's blood glucose over time, obtained from a mathematical model, for example, a physiological model describing the assimilation of insulin by the user's body and its impact on their blood glucose. More particularly, processing and control unit 105 may be configured to, based on the injected insulin history and on the ingested carbohydrate history, and based on a predetermined mathematical model, determine a curve representative of the expected trend of the user's blood glucose over time, over a period to come called prediction period or prediction horizon, for example, a period from 1 to 10 hours. Taking this curve into account, processing and control unit 105 determines the insulin doses that should be injected to the user during the prediction period to come, so that the user's real blood glucose (as opposed to the blood glucose estimated based on the model) remains within acceptable limits, and in particular to limit risks of hyperglycemia or of hypoglycemia.

As a variant, processing and control unit 105 may be configured to, outside of meal periods, implement an automated blood glucose regulation method of decision matrix type to determine the insulin doses to be delivered to the patient, according to various observed parameters such as the current blood glucose level measured by sensor 101, or also the blood glucose variation speed (or slope) over a past period.

In another variant, processing and control unit 105 may be configured to, outside of meal periods, alternate between a regulation method of MPC type and a regulation method of decision matrix type.

In principle, the user declares each of their meals, and in particular the meal ingestion time and the approximate amount of glucose ingested during the meal (also called meal size), via user interface 107.

Processing and control unit 105 is configured to, when a meal is declared by the user, activate a specific meal management module, this module implementing a regulation method adapted to taking into account the physiological specificities linked to the assimilation of a meal. The meal management module is for example implemented in software form by means of processing and control unit 105. The regulation method implemented by the meal management module may comprise a step of calculation, based on the data entered by the user, and particularly on the declared time and the declared size of the meal, of an insulin bolus, that is, a complementary insulin dose to be injected to the user as a supplement to a normally-injected basal insulin rate. The meal management module then controls the bolus injection by injection device 103. The bolus may be administered in a single injection or in a plurality of successive injections, for example, in two injections. The bolus may be administered at the time of the declaration of the meal, or at the beginning of the meal or even little before the beginning of the meal if the declaration occurs before the beginning of the meal. If the meal is announced with a delay, the bolus may be administered after the beginning of the meal. As an example, the insulin bolus injected during a meal may be at least twice greater than the insulin dose normally injected within one hour outside of meal periods. As an example, the basal insulin rate normally injected to the user outside of meal periods is in the range from 0.3 to 1.5 UI/h, where UI designates an international insulin unit, that is, the biological equivalent of approximately 0.0347 mg of human insulin. The bolus determined by the meal management module and then injected by injection device 103 is for example in the range from 3 to 30 UI according to the declared size of the meal and to the subject's sensitivity to insulin. After the injection of the bolus, the meal management module can modulate the basal insulin rate injected to the user, for example by means of a PID filter or PID (“Proportional Integral Derivative”) corrector, for a predetermined time period, for example, a period of three hours following the bolus injection, to bring the current blood glucose back to a target value. At the end of this modulation period, the meal management method ends. Processing and control unit 105 can then implement another blood glucose regulation method, for example, a method of MPC type or of decision matrix type such as described hereabove.

A limitation of the above-described operation is that its efficiency strongly depends on the user's declarations. If the user omits to declare a meal, the meal management module is not activated. The user's blood glucose is then ensured by a regulation method non-specific to meals, for example, a method of MPC type or of decision matrix type such as described hereabove, which may result in relatively long hyperglycemia periods, particularly due to the lack of aggressiveness of these methods towards situations of hyperglycemia linked to a meal ingestion.

FIG. 2 is a diagram illustrating an example of a blood glucose regulation system adapted to managing meals non-declared by the user. This method is based on the use of statistical data generated by training based on a history of data concerning the user.

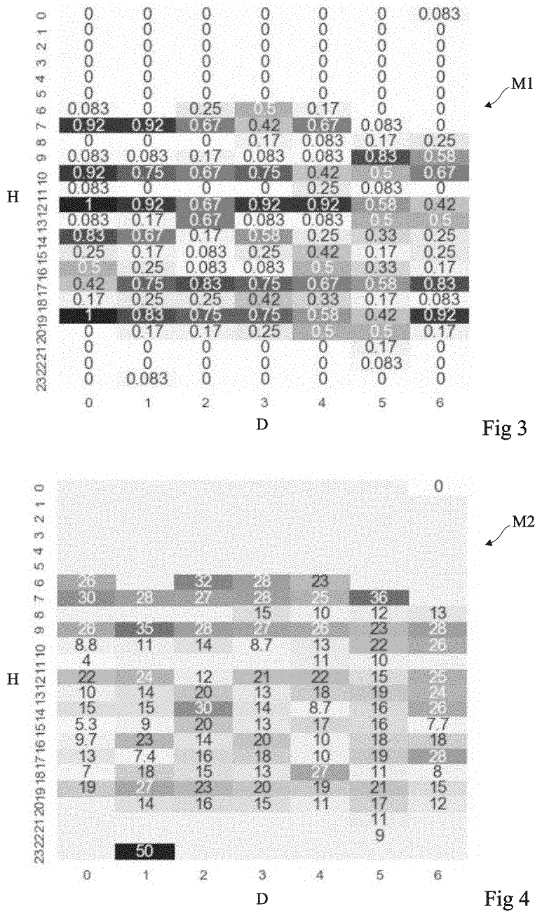

The method of FIG. 2 is more particularly based on the use of a meal probability table or matrix M1, of the type illustrated in FIG. 3, and of an average meal size table or matrix M2, of the type illustrated in FIG. 4.

In the example of FIG. 3, table M1 comprises an integer number H of rows and an integer number D of columns. In the shown example, number D is equal to 7, each column of table M1 corresponding to a day in the week. Further, in this example, number H is equal to 24, each row of table M1 corresponding to an hour of the day.

In the example of FIG. 4, table M2 comprises the same number H of rows and the same number D of columns as table M1. Each column of table M2 corresponds to a day in the week and each row of table M2 corresponds to an hour of the day.

Designating by d the rank of the columns of tables M1 and M2, d being an integer ranging from 0 to 6, and by h the rank of the rows of tables M1 and M2, h being an integer ranging from 0 to 23, each value M1(d, h) of coordinates (d, h) in table M1 corresponds to the probability for a meal to have been taken by the user on day d within time slot h, and each value M(d, h) of coordinates (d, h) in table M2 corresponds to the average size of the meals generally taken by the user on day d within time slot h, for example, in gCHO, that is, in grams of carbohydrates. In this example, the days of rank d=0 to d=6 respectively correspond to the seven days of the week, and each time slot of rank h corresponds to a one-hour slot, from time h to time h+1.

Tables M1 and M2 may be generated by training based on a history of data acquired for the user during a previous training phase, for example, a phase of from a plurality of days to a plurality of weeks. As an example, each value M1(d, h) of table M1 corresponds to the percentage of times that a meal has been declared by the user on day d and within slot h during the training phase. Each value M2(d, h) of table M2 for example corresponds to the averages size of the meals declared by the user on day d and within slot h during the training phase.

Tables M1 and M2 may be stored in a memory circuit of processing and control device 105.

Tables M1 and M2 may for example be updated along the use of the system, each time the user declares a meal via user interface 107.

When personalized tables M1 and M2 are not available for a given subject, one may, as a first approach, use generic tables M1 and M2, obtained from a population data history. Generic tables M1 and M2 may for example be determined for several types of population, for example, schooled children, teenagers, adults, possibly with different meal rates, for example, according to the country of residency.

The method of FIG. 2 comprises a step 201 (DET) of detection of an event likely to correspond to a meal non-declared by the user. As an example, the detection implemented at step 201 may be based on measurements provided by the blood glucose sensor 101 of the system. An event detected at step 201 for example corresponds to a rise in the user's blood glucose of the type normally observed after a food ingestion, but not explained by a prior declaration of a meal by the user. As an example, processing and control unit 105 may be configured to implement a continuous monitoring of the user's blood glucose curve in order to detect such events.

When, at a time t0, an event is detected at step 201, a first step 203 (USR1) of interrogation of the user by means of user interface device 107 is implemented. During this step, it is asked to the user, via device 107, whether they have taken a meal within a period T_ANT of predetermined duration preceding time t0, for example, within the three hours preceding time t0.

If, at step 203, the user answers, via device 107, that they have not (N) taken an undeclared meal within the considered period T_ANT, the regulation carries on at a step 205 (REGUL) with a regulation method non-specific to meals, for example, a method of MPC type or of decision matrix type such as described hereabove.

If, at step 203, the user answers that they have (Y) taken an undeclared meal within the considered period T_ANT, a second step 207 (USR2) of interrogation of the user by means of user interface device 107 is implemented. During this step, it is asked to the user, via device 107, the size of the undeclared meal and the undeclared meal ingestion time. In other words, during steps 203 and 207, it is asked to the user to declare ex post facto the meal that they had omitted to declare.

If, at step 207, the user answers (A), via device 107, by indicating the size and the meal of the undeclared meal, processing and control unit 105 activates a specific meal management module, this module implementing, during a step 209 (PMM), a regulation method adapted to taking into account the physiological specificities linked to the assimilation of a meal, by taking into account the size of the meal and the time of the meal declared by the user.

If, at step 203, the use does not answer (NA) to the asked question concerning the possible ingestion of an undeclared meal within period T_ANT, a step 211 (PMS1) is implemented, during which processing and control unit 105 determines, based on table M1, whether the probability for an undeclared meal to have been ingested by the user within period T_ANT is greater or not than a predetermined threshold TH. For this purpose, processing and control unit 105 determines whether matrix M1 contains a probability value M1(d, h) greater than threshold TH in the column of rank d corresponding to the current day and in the rows corresponding to the smallest time range comprising period T_ANT.

If, at step 211, it is determined that table M1 comprises no meal ingestion probability value M1(d, h) greater than threshold TH within range T_ANT, step 205 is implemented, that is, the regulation carries on with a regulation method non-specific to meals, for example, a method of MPC type or of decision matrix type such as described hereabove.

If, at step 211, it is determined that table M1 comprises a meal ingestion probability value M1(d, h) greater than threshold TH within range T_ANT, a step 213 (PMS2) is implemented, during which processing and control unit 105 determines an estimated time slot of meal ingestion by the user within period T_ANT, and an estimated size of the ingested meal.

To determine the estimated time slot of meal ingestion by the user, processing and control unit 105 may use table M1. The estimated time slot for example corresponds to the slot of coordinates d, h of period T_ANT for which the meal ingestion probability value M1(d, h) of table M1 is the highest. As a variant, the meal may be positioned at the time when the event has been identified at step 201.

To determine the estimated size of the ingested meal, processing and control unit 105 may use table M2. The estimated size of the meal for example corresponds to value M2(d, h) of table M2 in the time slot of coordinates d, h estimated based on table M1. As a variant, the estimated size of the meal may be determined based on the observed blood glucose rise.

At the end of step 213, step 209 is implemented, that is, processing and control unit 105 activates a specific meal management module, this module implementing a regulation method adapted to taking into account physiological specificities linked to the assimilation of a meal. In this case, the regulation method implemented at step 209 takes as input parameters the estimated size and the estimated time slot of the meal determined at step 213 based on tables M1 and M2.

If, at step 207, the user does not answer (NA) to the question asked regarding the time and the size of the undeclared meal taken during period T_ANT, step 213 is implemented to estimate the time slot and the size of the undeclared meal based on tables M1 and M2. Step 209 is then implemented similarly to what has just been described.

Preferably, the insulin bolus to be injected to the user, determined by the regulation method implemented at step 209, is weighted by an aggressiveness factor, which factor may take a first value when step 209 is implemented after a meal declaration performed by the user at step 207, and a second value smaller than the first value when step 209 is implemented after a meal size and time estimation based on tables M1 and M2 at step 213. In the case where step 211 is implemented, the second value may be all the lower as the probability for a meal to have been taken by the user within period T_ANT is low.

The method described in relation with FIG. 2 advantageously enables, by using statistical data representative of the times and sizes of the meals usually taken by the user, to process as meals, by means of a specific regulation module, events likely to correspond to meals but non-declared as such by the user.

As a variant, steps 203 and 207 of interrogation of the user may be omitted. In this case, when an event likely to correspond to a meal is detected at step 201, step 211, is directly implemented, followed by step 205 if it is determined at step 211 that the probability for a meal to have been taken by the user during the previous period T_ANT preceding time t0 of detection of the event is smaller than threshold TH, or followed by step 213 and then by step 209 in the opposite case.

Various embodiments and variants have been described. Those skilled in the art will understand that certain features of these various embodiments and variants may be combined, and other variants will occur to those skilled in the art. In particular, the described embodiments are not limited to the examples described in relation with FIGS. 3 and 4 of time division granularity of the horizontal and vertical axes of tables M1 and M2. As a variant, rather than having one meal probability value and one average meal size value per hour and per day over seven consecutive days in tables M1 and M2, tables M1 and M2 comprising one meal probability value and one average meal size value per hour and per working day (independently from the considered day) and one meal probability value and one average meal size value per hour and per non-working day (independently from the considered day) may be provided. More generally, any other division adapted to the user's lifetime may be considered.

Further, the described embodiments are not limited to the example described in relation with FIG. 2 where the detection implemented at step 201 is based on blood glucose measurements delivered by sensor 101. More generally, the detection of events likely to correspond to meals implemented at step 201 may be based on any other appropriate indicator, as a complement to or to replace blood glucose measurements.

1. Automated blood glucose regulation system, comprising:
a blood glucose sensor;
an insulin injection device; and
a processing and control unit,
wherein the processing and control unit is configured to implement a method of management of undeclared meals, this method comprising the steps of:
a) detecting, at a time t0, an event likely to correspond to a meal non-declared by a user;
b) when an event is detected at step a), determining, based on a first table generated by training from a history of the user's data, a probability for a meal to have been taken by the user within a period T_ANT of predetermined duration preceding time t0;
c) if the probability determined at step b) is greater than a threshold TH, determining, based on the first table, an estimated time slot of meal ingestion by the user within period T_ANT and, based on a second table generated by training from a history of the user's data, an estimated size of the meal, and then activating a meal management module of the regulation system and transmitting to said module the estimated time slot and size of the meal.

2. System according to claim 1, wherein:
the first table is formed of a series of probability values, each corresponding to a percentage of times that a meal has been declared by the user within a determined time interval of a determined time cycle, during a training phase comprising a plurality of occurrences of said time cycle; and
the second table is formed of a series of meal size values, each corresponding to the average meal size declared by the user within each time interval of said time cycle during the training phase.

3. System according to claim 2, wherein the first time cycle is divided into a plurality of time intervals, the number of values of the first table and the number of values of the second table being equal to the number of time intervals of said time cycle.

4. System according to claim 1, wherein the processing and control unit is configured to, if the probability determined at step b) is lower than threshold TH, implement a blood glucose regulation method non-specific to meals.

5. System according to claim 1, further comprising a user interface device coupled to the processing and control unit.

6. System according to claim 5, wherein the processing and control unit is configured to, when an event is detected at step a), implement, before step b), a first step of interrogation of the user, by means of the user interface device, to ask them whether they have had an undeclared meal within period T_ANT.

7. System according to claim 6, wherein the processing and control unit is configured to:
if the user answers negatively to the first interrogation, implement a blood glucose regulation method non-specific to meals;
if the user answers positively to the first interrogation, implement a second step of interrogation of the user, by means of the user interface device), to ask them the time and the size of said undeclared meal taken during period T_ANT; and
if the user does not answer to the first interrogation, implement step b) and then step c).

8. System according to claim 7, wherein the processing and control unit is configured to:
if the user answers to the second interrogation, activate the meal management module of the regulation system and transmit to said module the time and the size declared by the user as an answer to the first interrogation; and
if the user does not answer to the second interrogation, determine, based on the first table, an estimated time slot of meal ingestion by the user within period T_ANT and, based on the second table, an estimated size of the meal, and then activate the meal management module of the regulation system and transmit to said module the estimated time slot and size of the meal.

9. System according to claim 1, wherein the processing and control unit is configured to, at step c), determine, by means of the meal management module, an insulin bolus to be injected to the user according to the estimated meal size.

10. System according to claim 9, wherein the processing and control unit is configured to, at step c), weigh said bolus by a coefficient which is a function of the probability determined at step b).

11. System according to claim 1, wherein the first and second tables are stored in a memory circuit of the processing and control unit.
